(19) United States
(12) Patent Application Publication
(10) Pub. No.: US 2003/0219809 A1
(43) Pub. Date: Nov. 27, 2003

(54) Surface plasmon resonance shifting interferometry imaging system for biomolecular interaction analysis

(75) Inventors: Shean-Jen Chen, Taoyuan (TW); Feng-Ming Hsiu, Ilan (TW); Chien-Hung Tsai, Tainan (TW); Chia-Yuan Tsou, Taipei (TW); Chi-Meng Tzeng, Taipei (TW)

Correspondence Address: COHEN, PONTANI, LIEBERMAN & PAVANE, 551 FIFTH AVENUE, SUITE 1210, NEW YORK, NY, 10176, US

(73) Assignee: U-Vision Biotech, Inc.

(21) Appl. No.: 10/397,770

(22) Filed: Mar. 26, 2003

Related U.S. Application Data
(60) Provisional application No. 60/367,890, filed on Mar. 26, 2002.

(51) Int. Cl. C12Q 1/68; G01N 21/41

(52) U.S. Cl. 435/6; 356/134

(57) ABSTRACT

A novel surface plasmon resonance (SPR) imaging system based on modified Mach-Zehnder phase-shifting interferometry (PSI) measures the spatial phase variation of a resonantly reflected light during chemical or biological detection. The SPR microarray can diagnose the target analyte without additional labeling in the real-time analysis. Experimental results demonstrate that the detection limit of the SPR PSI imaging system is improved to about 1 pg/mm² surface coverage of chemical or biological material for each individual spot over that of the conventional SPR imaging system. Therefore, the SPR PSI imaging system and its SPR microarray can provide the capability of real-time analysis, with high resolution and at high-throughput screening rates.

### RELATED APPLICATIONS

This application claims priority from U.S. Provisional Patent Application Serial No. 60/367,890 which was filed on Mar. 26, 2002.

### BACKGROUND OF THE INVENTION

1. Field of the Invention

The present invention is related to an interferometric method and apparatus for detection of the spatial phase variation of reflected light under surface plasmon resonance (SPR) conditions due to refractive index changes. The interferometric method and apparatus can be successfully applied in biomolecular interaction analysis.

2. Description of the Related Art

SPR has become a promising technique because of its extreme sensitivity to plasmon resonance from environmental changes without the need for biomolecular labeling. Through the use of biospecific capturing of molecules immobilized on the surface of the biosensor, the SPR biosensors have the ability of detecting biomaterial concentration, thickness, and binding kinetic data for specific biological analytes. With this approach, the biomolecular interaction analysis (BIA) applications of SPR systems such as antigen-antibody interaction, receptor-ligand interaction, DNA-protein interaction, and DNA hybridization have dramatically increased since 1990.¹⁻³

The SPR sensor is based on a coupling prism, coated with a metal thin film. The metal film is typically about 50 nm-thick Au or Ag for providing the optimum resonance condition in the near infrared and visible light regions. The surface plasmon, a charge density wave propagating along the interface between metal and dielectric medium, absorbs the energy of the evanescent wave from the incident P-wave light that would be totally reflected without the metal film, and therefore, the reflectivity of the incident light is decreased. This excitation method is called attenuated total reflection (ATR).⁴ The phenomenon of SPR can be observed as an absorption spectrum in the dependence of the intensity of P-wave light reflected from the metal film on the incident angular or wavelength interrogation. The SPR sensors based on wavelength interrogation are less precise than the SPR sensors applying angular interrogation due to the limitation in the resolving power (λ/Δλ) of the optical spectrum analyzer. When the resolving power achieved is better than 10⁵ in the near infrared and visible light regions, the wavelength interrogation will be prevalent owing to its more compact optical system. With the SPR biosensor, biomolecular interaction at the interface is detected by a change in the refractive index or layer thickness at the biosensor surface. Therefore, the resonant angular shift based on angular interrogation can be promptly detected while the condition of surface plasmon excitation is satisfied.⁵⁻⁶ Most widely used configuration of SPR biosensors is based on prism-coupler ATR system that has four main detection approaches: intensity measurement, angular interrogation, wavelength interrogation, and phase measurement. Experimentally, both the resolution of angular interrogation and phase measurement can achieve about 1 pg/mm² surface coverage of the biomaterial.

The commercially successful “BlAcore” SPR system (BlAcore AB, Uppsala, Sweden) based on angular interrogation has been used previously to analyze the biomolecular interaction, but with no imaging capability (high-throughput screening).⁷ The worldwide trend concerning advanced SPR biosensors and systems are not only to improve the sensitivity, resolution, stability, and speed, but also to achieve high-throughput screening capability. Therefore, to achieve this high-throughput screening capability and also to keep the detection limit of 1 pg/mm², SPR microarrays and their detection systems are needed. In the conventional SPR imaging system, a collimated light source is used to illuminate a coupling prism or grating with thin gold film sample assembly at an incident angle that is near to the SPR angle, and the intensity variation of the reflected light is detected at a fixed angle with an area scan charge coupled device (CCD) camera to produce an SPR image. Even if these SPR imaging systems are capable of high-throughput screening, it is hard to detect low concentrations of low molecular weight analytes.⁸⁻¹¹

### SUMMARY OF THE INVENTION

Therefore, one object of the present invention is to develop a convenient and accurate SPR imaging system to observe chemical or biological interactions.

A further object is to increase the resolution of the SPR imaging system for microarray screening, and achieve high-throughput screening capability by measuring the spatial phase of a resonantly reflected light.

In accordance with the present invention, we provide a method of detecting a spatial phase variation of light resonantly reflected from a sensor comprising a metallic film having opposing sides. The method comprises the following steps:

directing light onto one side of the metallic film to produce the light resonantly reflected from the sensor;

splitting the resonantly reflected light into a measuring p-wave and a reference s-wave;

combining the measuring p-wave and the reference s-wave to produce an interference pattern; and

recording the interference pattern to detect the spatial phase variation of the light resonantly reflected from the sensor.

The method in accordance with the present invention may further comprise a step of producing a phase shift of a wave selected from the group consisting of the reference s-wave, the measuring p-wave and combination thereof. The phase shift may be π/2.

To facilitate producing the interference pattern, the method in accordance with the present invention may further comprise a step of rotating the reference s-wave into a reference p-wave before the step of combining. For example, a half-wave plate may be used for this function. Similarly, the method may comprises a step of rotating the measuring p-wave into a measuring s-wave before the step of combining. Another embodiment to facilitate the interference pattern may be a step of passing at least part of the measuring p-wave and at least part of the reference s-wave at the same oscillating direction, after the step of combining but before the step of recording. Preferably, the step of passing may be conducted by using a fixed polarization analyzer having an optical axis at an angle of 45 degree with respect to the incident plane of the reference s-wave and the measuring p-wave.

In accordance with another embodiment of the present invention, we provide an apparatus for measuring a spatial phase variation of resonantly reflected light. The apparatus comprises:

a sensor comprising a metallic film;

a light source disposed over the sensor for directing light onto the sensor to produce the resonantly reflected light from the sensor;

a polarization beam splitter disposed along the propagating path of the resonantly reflected light for splitting the resonantly reflected light into a reference s-wave and a measuring p-wave;

means for combining the reference s-wave and the measuring p-wave to produce an interference pattern; and

an interference pattern detector disposed along a path where the combined reference s-wave and the measuring p-wave propagate to determine the spatial phase variation of the light resonantly reflected from the sensor.

The apparatus in accordance with the present invention may further comprise a phase shifter disposed along the propagating path of the reference s-wave or the measuring p-wave. The apparatus may also comprise at least two phase shifters, so that at least one phase shifter is disposed at the propagating path of the reference s-wave and the measuring p-wave respectively.

In the apparatus in accordance with the present invention, the means for combining comprises a mirror, a first beam splitter and a second beam splitter, and the polarization beam splitter, the mirror, the first beam splitter, and the second beam splitter are disposed in Mach-Zehnder format. Hence, the polarization splitter and the first beam splitter defines a first arm, the mirror and the second beam defines a second arm, the p-wave propagated along the first arm and the s-wave propagated along the second arm; the first splitter divides the p-wave into a transmitted p-wave and a first reflected p-wave which enters the second splitter and forms a second reflected p-wave; the s-wave from the polarization splitter is reflected by the mirror to the second beam splitter and forms a transmitted s-wave which is combined with the second reflected p-wave.

In accordance with yet another embodiment of the present invention, the method of detecting a spatial phase variation of resonantly reflected light may comprises the following steps:

splitting light into a measuring wave and a reference wave;

directing the measuring wave onto a sensor comprising a metal film to produce the light resonantly reflected from the sensor;

producing a phase shift of a wave selected from the group consisting of the reference wave, the measuring wave and combination thereof;

combining the measuring wave and the reference wave to produce a interference pattern; and

recording the interference pattern to determine the spatial phase variation of the light resonantly reflected from the sensor.

Preferably, the wave to be phase shifted may be the reference wave.

The various features of novelty which characterize the invention are pointed out with particularity in the claims annexed to and forming a part of the disclosure. For a better understanding of the invention, its operating advantages, and specific objects attained by its use, reference should be had to the drawing and descriptive matter in which there are illustrated and described preferred embodiments of the invention.

Other objects and features of the present invention will become apparent from the following detailed description considered in conjunction with the accompanying drawings. It is to be understood, however, that the drawings are designed solely for purposes of illustration and not as a definition of the limits of the invention, for which reference should be made to the appended claims. It should be further understood that the drawings are not necessarily drawn to scale and that, unless otherwise indicated, they are merely intended to conceptually illustrate the structures and procedures described herein.

### BRIEF DESCRIPTION OF THE DRAWINGS

In the drawings:

FIG. 1 shows a Kretschmann configuration of the SPR biosensors.

FIG. 2 shows the simulated reflectivity and phase shift curves of the reflected light of an SPR sensor with the configuration BK7/Au film/biomaterial/water with dielectric constants of 1.515²/−10.8+1.47j/2.01+0.22j/1.33².

FIG. 3 shows a typical SPR DNA microarray in accordance with one embodiment of the present invention.

FIG. 4 presents the SPR PSI imaging system in accordance with one embodiment of the present invention.

FIG. 5 shows the time dependence of the phase shift of the SPR PSI imaging system in the sequential cyclic replacement of 100% N₂ with 100% Ar per 9 minutes.

FIG. 6 shows five interference frames for 28 thiol/ssDNA spots in a 6×8 mm² area-scan CCD camera with 640×480 pixels.

FIG. 7 shows SPR PSI data analysis for 6 thiol/ssDNA spots.

### DETAILED DESCRIPTION OF THE PRESENTLY PREFERRED EMBODIMENTS

1. Excitation of Surface Plasmon Resonance by Light

The SPR is an optical phenomenon in which incident P-wave light excites a surface plasmon wave (SPW), i.e. a surface charge density wave at the interface of a thin metal and dielectric sample medium, to reach a resonance condition. Excitation of SPR can occur when the parallel component of the wave vectors of the incident light kₓ¹ and the wave vector of SPW k_{spw} satisfy the matching condition,⁴

kₓ¹=kₒ\sqrt{ε₀} sin θ=k_{spw},

where θ is the incident angle of light and ε₀ is the wavelength dependent dielectric constant of the prism. Then, the wave vector of SPW can be approximated as

$${k}_{spw} ≅ {k}_{o} \sqrt{\frac{{ϵ}_{1} {ϵ}_{2}}{{ϵ}_{1} + {ϵ}_{2}}} , \qquad ( 2 )$$

where ε₂ and ε₁ are the wavelength dependent complex dielectric constants of the dielectric sample and the metal, respectively. When the matching condition is satisfied, most of the incident light energy will be transferred to the surface plasmon, in other words, most of the incident light will be absorbed by the excitation of surface plasmon wave, resulting in an attenuated reflected spectrum.

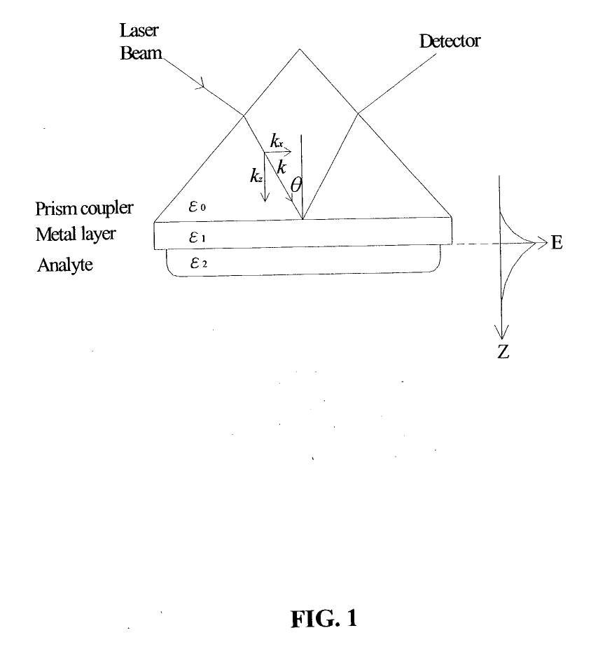

Most widely used configuration of SPR sensors are based on prism coupler-based SPR system (ATR), as shown in FIG. 1. The configuration basically consists of a prism, a thin metal film, and the dielectric layer in which the interested immobilized analyte to be probed. Theoretical results using Fresnel's equations demonstrate that the reflectivity minimum (strong absorption) only occurred on P-wave. The reflection spectrum corresponds to the molecular concentration and thickness of the biomaterials. The quantitative description of the reflectivity R for the P-wave can be given by Fresnel's equations for the three-layer or more system as

$$R ≡ {\vert {r}_{012}^{p} \vert}^{2} = {\vert \frac{{r}_{01}^{p} + {r}_{12}^{p} exp ( 2 ⅈ {k}_{z1} d )}{1 + {r}_{01}^{p} {r}_{12}^{p} exp ( 2 ⅈ {k}_{z1} d )} \vert}^{2} , \qquad ( 3 )$$

where the thickness of the metal film is d and the reflection coefficient of the P-wave is given as

$${r}_{i k}^{p} = ( \frac{{k}_{zi}}{{ϵ}_{i}} - \frac{{k}_{z k}}{{ϵ}_{k}} ) / ( \frac{{k}_{zi}}{{ϵ}_{i}} + \frac{{k}_{z k}}{{ϵ}_{k}} ) . \qquad ( 4 )$$

Moreover, r₀₁₂^{p}=Re(r₀₁₂^{p})+Im(r₀₁₂ ^{p}). Hence, the phase of the reflected light can be given as

φ=tan⁻¹(r₀₁₂^{p})=tan⁻¹(Im(r₀₁₂^{p})/Re(r₀₁₂^{p})). (5)

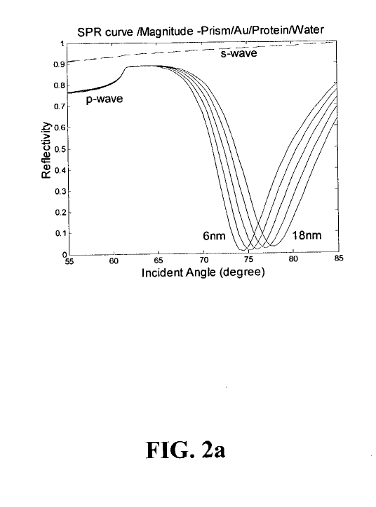

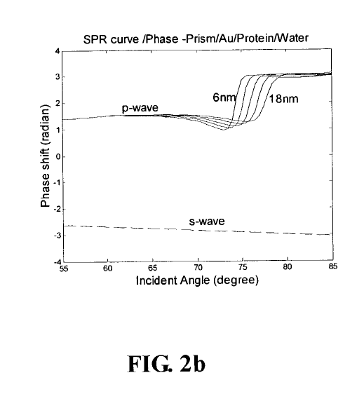

FIG. 2 shows the simulated reflectivity and phase shift curves of the reflected light of an SPR sensor with the configuration BK7/Au film/biomaterial/water with dielectric constants of 1.515²/−10.8+1.47j/2.01+0.22j/1.33². The thickness variations of biomaterial films are from 6 to 18 nm per 3 nm. The solid lines for the P-wave show the minimum reflectivity and phase jump in the SPR angle. The dashed lines for the S-wave are constant.

2 SPR Microarray and its Imaging System

Molecular biologists conventionally employ the method of hybridization: difference sequence DNA oligonucleotides with several tens to hundreds of nucleotides (nt), which is complementary to the sequence of interest from labeled target DNA. This is a relatively time taking system because a fluorescence material is used to label target DNA. In contrast, the major principles of SPR to detect the biomolecular interaction are to first immobilize probe DNA or antibody on the SPR sensors, and then measure the change of the resonant condition in real time when unlabelled target DNA or antigen interacts with the probe DNA or antibody. The biomolecular interaction can be characterized and quantified and the kinetic BIA can be studied.

2.1 Design and Fabrication of SPR DNA Microarray

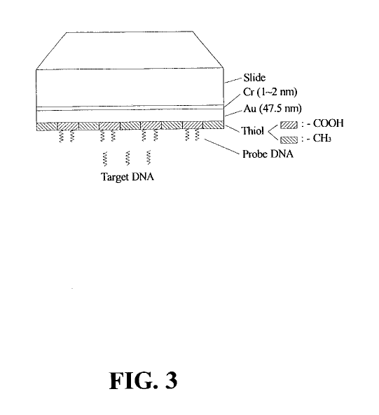

To avoid the chemical reaction with metal film during DNA immobilization process, gold is selected a better candidate than silver. The poor adhesion of gold to glass is a problem, so a very thin chromium film (1˜2 nm) is deposited to the glass as an underlay. In order to achieve the optimum sensitivity, the gold film deposited must have the thickness of 47.5 nm with 1 nm accuracy according to Eq. (3) and the roughness is controlled less than 10 Årms. Array biosensing is applied to achieve the high throughput screening capability. In order to bind the probe DNA on the gold film slide, the slide is merged in a 1-mM thiol solution (HS(CH₂)₁₅COOH) over 6 hours, and then is placed inside EDC/MES solution (EDC of 100 mg merged in 40-mM MES of 100 ml) over 6 hours. After cleaning the slide with D.I. water and alcohol, the probe DNA is mechanically spotted with matrix. Finally, the blocking solution (methanol) is applied to modify the functional group of thiol —COOH into —CH₃ to avoid capturing the target DNA on the free thiol/EDC area. With the above process, SPR DNA microarray is designed and fabricated, as shown in FIG. 3. Thus the SPR imaging system observes simultaneously the DNA microarray hybridization to achieve the high-throughput screening capability.

2.2 SPR PSI Imaging System

In accordance with the present invention, the novel SPR imaging system is based on modified Mach-Zehnder PSI technique for BIA, which measures the spatial phase variation of a resonantly reflected light in the biomolecular interaction. Since the analysis required for PSI is not dependent on finding fringe centers or following fringes, any kind of fringe pattern can be analyzed. This system is more practical and accurate than other SPR phase imaging systems. The SPR microarray designed with over a thousand different biomolecular spots (such as DNA, protein, or receptors.) can be detected simultaneously under the SPR PSI imaging system. Therefore, the detection sensitivity of the SPR PSI imaging system is expected to achieve 1 pg/mm² surface coverage of biomaterial in each individual spot over conventional SPR imaging systems.

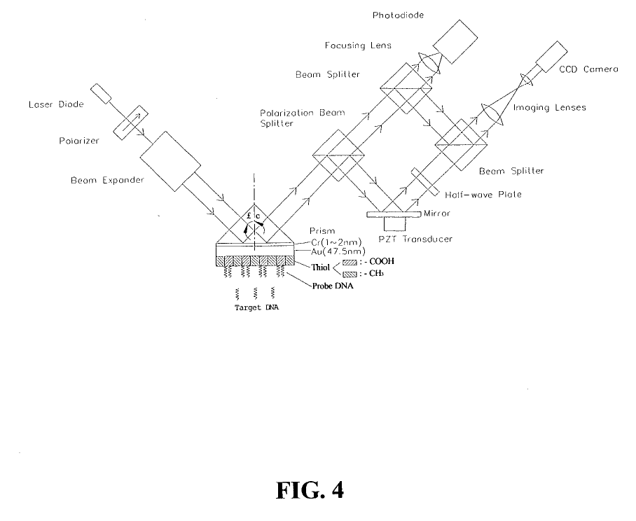

FIG. 4 presents a SPR PSI imaging system in accordance with one embodiment of the present invention. First, rotating the incident angle of a single wavelength beam onto the SPR microarray to detect the reflecting intensity variation, the incident angle corresponding to the minimum reflectivity is defined as the SPR angle of a new model. Near the SPR angle, the collimated P-wave and S-wave (typically He—Ne laser or laser diode) is passed through a prism to excite a slide with deposited thin gold film and biomaterial. The SPW is excited only by the P-wave at the SPR angle, so the phase of P-wave is dramatically changed during biomolecular interaction. Therefore, the P-wave corresponds to the actual signal and the S-wave is the reference signal. The phase differences between the P-wave and S-wave measured by using the modified Mach-Zehnder PSI system at various locations create interference patterns and are converted to analyze the biomolecular interaction by using linear data analysis and Fresnel's calculations. The modified Mach-Zehnder PSI system splits the reflected light from the SPR microarray into P-wave and S-wave, and optical phase shifts by using PZT transducer mirror are introduced into the S-wave among each of the sequentially recorded 5 interferograms through a CCD camera.¹⁶ The phase can be reconstructed based on the following Eq. (6) and two-dimensional (2-D) phase unwrapping algorithms.¹⁷

$$φ ( x , y ) = {tan}^{- 1} [ \frac{2 ( {I}_{2} - {I}_{4} )}{2 {I}_{3} - {I}_{5} - {I}_{1}} ] , \qquad ( 6 )$$

where Iᵢ is one of the five interferograms, i=1, 2, . . . , 5. After phase reconstruction, a 2-D data of the measured phase in terms of biomolecular interaction is established and then converted to characterize the biomolecular interaction.

These interferograms are recorded by varying the reference phase. The wavefront phase modulo 2π is then calculated at each measurement point as the arctangent of a function of the interferogram intensities measured at that individual spot. Therefore, the final wavefront map is then obtained by unwrapping the phases to remove the 2π phase discontinuities. A five-step reconstruction algorithm is used, because it can overcome the unknown sensitivity of the PZT.¹⁶⁻¹⁷

3 Experimental Results

Two experiments are setup up to verify the performance (such as resolution, accuracy, and stability.) of SPR PSI imaging system. First, the difference refractive index Δn (1.5×10⁻⁵) of N₂ and Ar is precisely demonstrated to prove the global high accuracy of the system. Then, the high-throughput capability of the system is shown in DNA microarray hybridization.

3.1 Global Test

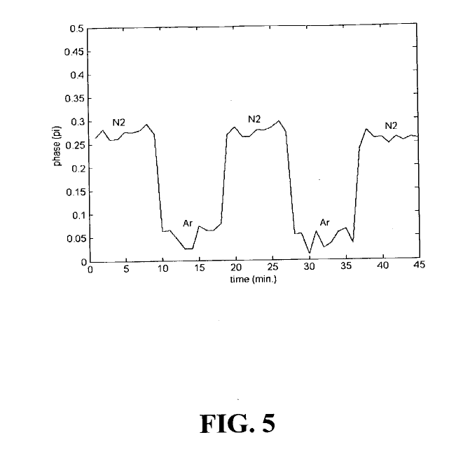

The preliminary experiment convinces that the SPR PSI imaging system is workable when the refractive indices of 100% N₂ and 100% Ar are notably separated. Difference refractive index Δn is about 1.5×10⁻⁵ under controlled conditions. These gases entered the flow system through an extended temperature controlled chamber that controls the temperature variation below 0.1° C. and the flow rate 50 ml/min. FIG. 5 shows the time dependence of the phase shift of the SPR PSI imaging system in the sequential cyclic replacement of 100% N₂ with 100% Ar per 9 minutes. The phase difference between N₂ and Ar is measured as 0.22 π. After testing the short-term stability and long-term drift, it is found that the SPR PSI imaging system can achieve the global resolution better than π/300 with 20×20 mm² area. Hence, the SPR PSI imaging system presents the high resolution of the refractive indices of 5×10⁻⁷, so the global detection resolution corresponding to biomolecular interaction can achieve about 0.5 pg/mm² surface coverage. The conventional SPR imaging system based on measuring the intensity variation is not able to detect a tiny change under the replacement of N₂ with Ar condition.

3.2 Microarray Test

The design and fabrication of the SPR DNA microarray according to Sec. 2.1 is used here. After the completion of SPR sensors with the sequential configuration of glass slide, chromium film, and gold film, probe DNA array is applied to provide the high-throughput screening capability. In order to immobilize the probe DNA array on the SPR sensor, the SPR sensor is merged in 1-mM thiol solution (HS(CH₂)₁₅COOH) over 6 hours based on the kinetic study of the thiol binding on the gold film. Then, the slide is placed inside EDC/MES solution (EDC of 100 mg merged in 40-mM MES of 100 ml) over 6 hours to let EDC bind with —COOH part of the thiol. After using D.I. water and alcohol to clean the slide, 1 μM 5-mer probe ssDNA is mechanically spotted with 320 spots on 25×25 mm² area. Methanol is applied to modify the functional group of thiol —COOH into —CH₃ to avoid target DNA captured on the free —COOH/EDC area. Finally, the SPR DNA microarray is done.

The SPR DNA microarray is placed in contact with the coupled prism of the SPR PSI imaging system with some matching oil. The target DNA will hybridize with the probe ssDNA if they are partially or completely complemental in DNA sequence. The flow cell was controlled at 28° C. with the temperature variation below 0.1° C. to reduce the noise level from the change of dielectric constant of gold film. According to Sec. 3.2, the SPR PSI imaging system found the SPR angle by using the SPR reflectivity spectrum from the intensity variation of the measurement photodiode vs. the incident angle. The modified Mach-Zehnder interferometry is introduced to record the interference pattern created according to the phase difference between the P-wave and the S-wave. The interference pattern will have local variation if the target DNA hybridizes with some spots of the probe ssDNA array. The PSI implements a PZT actuator to produce a sequential π/2 phase shift to the S-wave to create four more interference frames. It provides more accurate fringe analysis by using the five-step phase reconstruction algorithm.¹⁶

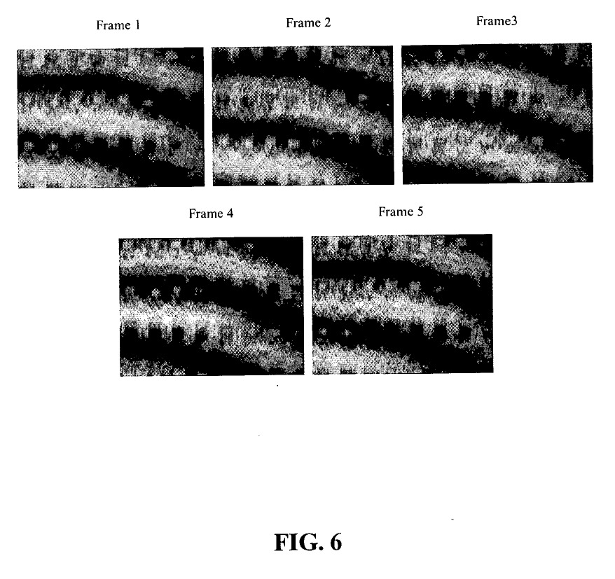

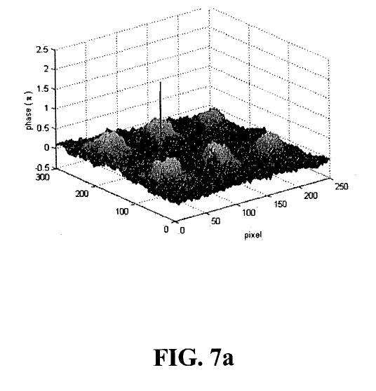

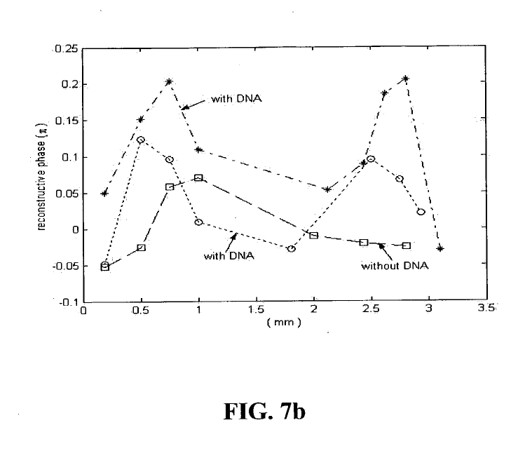

FIG. 6 shows five interference frames for 28 thiol/ssDNA spots in a 6×8 mm² area-scan CCD camera with 640×480 pixels. The spot size is of 0.5 mm diameter and the distance between probe ssDNA spots is 1 mm. If the part of the sensing area has the ssDNA spot, the local variation of the interference pattern can be observed, as shown in FIG. 6. Frame 5 is created based on Frame 1 to add four times of π/2 phase shift. Therefore, Frame 5 is similar to Frame 1 because of neglecting the influence of 2π phase shift. The phase difference is reconstructed with the five-step phase reconstruction algorithm, and then the biomolecular interaction is analyzed according to the phase difference by Fresnel's calculations. The reconstructed phase difference between with and without DNA sensing area is about 0.2 π, as shown in FIG. 7(a). FIG. 7(a) presents the phase jump of six probe ssDNA spots. The local resolution can achieve π/100 with 100×100 μm², thereby the screening area can be simultaneously monitored up to 2,500 individual spots in 10×10 mm² area to match high-throughput screening requirement in microarray DNA hybridization diagnostic by keeping the detection resolution about 1 pg/mm² surface coverage of biomaterial. The reconstructive phase variation in the 3 different scanning lines is shown in FIG. 7(b). The screening area with or without probe ssDNA can be distinguished. Therefore, the SPR DNA microarry can be detected simultaneously to meet the requirement of DNA sequence diagnostics in a single experiment. The SPR DNA microarray can be extensively applied to the BIA such as protein microarray. The SPR imaging system and its SPR DNA microarray can be used to observe DNA microarray hybridization in the real time, with high sensitivity, and at high-throughput screening rates.

Hence, the present invention provides a novel SPR PSI imaging system for the analyses of DNA hybridization without additional labeling. The SPR DNA microarray can simultaneously detect the target DNA to meet the requirement of DNA sequence diagnostic in a single experiment. Experimental results demonstrate that the detection limit for each individual spot under the SPR PSI imaging system can be achieved about 1 pg/mm². Based on these advantages, the SPR PSI imaging system and its SPR DNA microarray have been successfully used to observe DNA microarray hybridization in the real time, with high sensitivity and at high-throughput screening rates. Also, owing to the feasible and swift measurements, the SPR PSI imaging system can be extended to analyze biomolecular interaction.

The invention is not limited by the embodiments described above which are presented as examples only but can be modified in various ways within the scope of protection defined by the appended patent claims.

Thus, while there have shown and described and pointed out fundamental novel features of the invention as applied to a preferred embodiment thereof, it will be understood that various omissions and substitutions and changes in the form and details of the devices illustrated, and in their operation, may be made by those skilled in the art without departing from the spirit of the invention. For example, it is expressly intended that all combinations of those elements and/or method steps which perform substantially the same function in substantially the same way to achieve the same results are within the scope of the invention. Moreover, it should be recognized that structures and/or elements and/or method steps shown and/or described in connection with any disclosed form or embodiment of the invention may be incorporated in any other disclosed or described or suggested form or embodiment as a general matter of design choice. It is the intention, therefore, to be limited only as indicated by the scope of the claims appended hereto.

The entire contents of all the references cited in the preset invention are incorporated herein as reference.

### REFERENCES

1. B. Liedberg, C. Nylander, and I. Lundsr, “Surface plasmon resonance for gas detection and biosensing,” Sensor and Actuators B 4, 299-304 (1983).

2. Z. Salamon, M. E Brown, and G. Tollin, “Surface plasmon resonance spectroscopy: probing molecular interactions within membranes,” Trends in Biomedical Sciences 24, 213-219 (1999).

3. G. Steiner, V. Sablinskas, A. Hubner, C. Kuhne, R. Salzer, “Surface plasmon resonance imaging of microstructured monolayer,” J. Molecular Structure 509, 265-273 (1999).

4. H. Raether, Surface plasmons on smooth and rough surfaces and on gratings, Springer-Verlag, Berlin, 1988.

5. S.-H. Kim, K.-S. Ock, J.-H. Im, J.-H. Kim, K.-N. Koh, S.-W. Kang, “Photoinduced refractive index change of self-assembled spiroxazaine monolayer based on surface plasmon resonance,” Dye and Pigments 46, 55-62 (2000).

6. F. F. Bier, F. Kleinjung, and F. W. Scheller, “Real-time measurement of nucleic-acid hybridization using evanescent-wave sensors: steps towards the genosensor,” Sensors and Actuators B 38, 78-82 (1997).

7. http://www.biacore.com/

8. C. E. Jordan, R. M. Corn, “Surface plasmon resonance imaging measurement of electrostatic biopolymer adsorption onto chemically modified gold surface,” Anal. Chem. 69, 1449-1456 (1997).

9. http://www.htsbiosystems.com/

10. M. J. OBrien, V. H. Perez-Luna, S. R. J. Brueck, and G. P. Lopez, “A surface plasmon resonance array biosensor based on spectroscopic imaging,” Biosensors & Bioelectronics 16, 97-108 (2001).

11. B. P. Nelson, T. E. Grimsrud, M. R. Lies, R. M. Goodman, and R. Corn, “Surface palsmon resonance imaging measurements of DNA and RNA hybridization adsorption on DNA microarrays,” Anal. Chem. 73, 1-7 (2001).

12. A. V. Kabashin and P. I. Nikitin, “Surface plasmon resonance interferometer for bio- and chemical-sensors,” Optics Communications 50, 5-8 (1998).

13. P. I. Nikitin, A. N. Grigorenko, A. A. Beloglazov, M. V. Valeiko, A. I. Savchuk, O. A. Savchuk, G. Steiner, C. Kuhne, A. Huebner, and R. Salzer, “Surface plasmon resonance interferometry for micro-array biosensing,” Sensor and Actuators A 85, 189-193 (2000).

14. A. G Notcovich, V. Zhuk, and S. G Lipson, “Surface plasmon resonance phase imaging,” Appl. Phys. Lett. 76, 1665-1667 (2000).

15. F.-M. Hsiu, S.-J. Chen, C.-H. Tsai, et al., “Surface plasmon resonance imaging system with Mach-Zehnder phase-shift interferometry for DNA micro-array hybridization,” Proc. SPIE 4819, 167-174 (2002).

16. P. Hariharan, B. F. Oreb, and T. Eiju, “Digital phase-shifting interferometry: a simple error-compensating phase calculation algorithm,” Appl. Opt. 26, 2504-2505 (1987).

17. D. G. Ghiglia, G. A. Mastin, and L. A. Romero, “Cellular-automata method for phase wrapping,” J. Opt. Soc. Am. A 4, 267-280 (1987).

We claim:

1. A method of detecting a spatial phase variation of light resonantly reflected from a sensor comprising a metallic film having opposing sides, said method comprises:
directing light onto one side of the metallic film to produce the light resonantly reflected from the sensor;
splitting the resonantly reflected light into a measuring p-wave and a reference s-wave;
combining the measuring p-wave and the reference s-wave to produce an interference pattern; and
recording the interference pattern to detect the spatial phase variation of the light resonantly reflected from the sensor.

2. The method of claim 1 further comprising a step of producing a phase shift of at least one wave selected from the group consisting of the reference s-wave and the measuring p-wave.

3. The method of claim 2 wherein the phase shift is π/2.

4. The method of claim 2 wherein the wave is the reference s-wave.

5. The method of claim 1 further comprising a step of rotating the reference s-wave into a reference p-wave before the step of combining.

6. The method of claim 1 further comprising a step of rotating the measuring p-wave into a measuring s-wave before the step of combining.

7. The method of claim 1 further comprising a step of passing at least part of the measuring p-wave and at least part of the reference s-wave at the same oscillating direction, after the step of combining but before the step of recording.

8. The method of claim 7 wherein the step of passing is conducted by using a fixed polarization analyzer having an optical axis at an angle of 45 degree with respect to the incident plane of the reference s-wave and the measuring p-wave.

9. The method of claim 1 wherein the light is collimated.

10. The method of claim 1 wherein the light is polarized.

11. The method of claim 1 further comprising:
binding at least one probe onto the opposing side of the metallic film; and
introducing an analyte to the probe bound to the metallic film to determine the interaction between the probe and the analyte from the spatial phase variation.

12. The method of claim 11 wherein a plurality of probes are bound to the metallic film in a plurality of locations, the spatial phase variations at the plurality of locations are detected to determine the interactions between the probes and the analyte at the plurality of locations.

13. The method of claim 12 wherein the probes in at least two different locations are different from each other.

14. The method of claim 11 wherein the probe is DNA.

15. An apparatus for measuring a spatial phase variation of resonantly reflected light comprising:
a sensor comprising a metallic film;
a light source disposed over the sensor for directing light onto the sensor to produce the resonantly reflected light from the sensor;
a polarization beam splitter disposed along the propagating path of the resonantly reflected light for splitting the resonantly reflected light into a reference s-wave and a measuring p-wave;
means for combining the reference s-wave and the measuring p-wave to produce an interference pattern; and
an interference pattern detector disposed along a path where the combined reference s-wave and the measuring p-wave propagate to determine the spatial phase variation of the light resonantly reflected from the sensor.

16. The apparatus of claim 15 further comprising a phase shifter disposed along the propagating path of a wave selected from the group consisting of the reference s-wave and the measuring p-wave.

17. The apparatus of the claim 16 wherein the wave is the reference s-wave.

18. The apparatus of claim 15 wherein the means for combining comprises a mirror, a first beam splitter and a second beam splitter, and wherein the polarization beam splitter, the mirror, the first beam splitter, and the second beam splitter are disposed in the Mach-Zehnder format; whereby the polarization beam splitter and the first beam splitter defines a first arm, the mirror and the second beam defines a second arm, the measuring p-wave propagated along the first arm and the reference s-wave propagated along the second arm; the first splitter divides the measuring p-wave into a transmitted p-wave and a first reflected p-wave which enters the second splitter and forms a second reflected p-wave; the s-wave from the polarization splitter is reflected by the mirror to the second beam splitter and forms a transmitted s-wave which is combined with the second reflected p-wave.

19. The apparatus of claim 15 further comprising an intensity detector disposed along the path of the transmitted p-wave for detecting the intensity of the transmitted p-wave.

20. The apparatus of claim 15 further comprising a polarizer disposed along the propagating path of the light from the light source.

21. The apparatus of claim 15 further comprising a beam expander disposed along the propagating path of the light from the light source.

22. The apparatus of claim 16 wherein the phase shifter is a PZT-transducer mirror.

23. The apparatus of claim 18 wherein the mirror is the PZT transducer mirror.

24. The apparatus of claim 15 wherein the interference pattern detector is a CCD camera.

25. The apparatus of claim 15 further comprising a half-wave plate disposed along the propagating path of the reference s-wave.

26. The apparatus of claim 15 further comprising a half-wave plate disposed along the propagating path of the measuring p-wave.

27. The apparatus of claim 15 further comprising an analyzer disposed along the propagating path of the combined measuring p-wave and the reference s-wave.

28. The apparatus of claim 27 wherein the analyzer is a fixed analyzer having an angle of 45 degree with respect to the incident plane of the combined measuring p-wave and the reference s-wave.

29. The apparatus of claim 15 wherein the sensor further comprises a substrate coated with the metallic film.

30. The apparatus of claim 29 wherein the metallic film is gold, the substrate is a prism.

31. The apparatus of claim 30 wherein the sensor further comprises a thin chromium film deposited between the gold and the prism.

32. The apparatus of claim 15 wherein the light source is a laser diode.

33 A method of detecting a spatial phase variation of a resonantly reflected light comprising:
splitting light into a measuring wave and a reference wave;
directing the measuring wave onto a sensor comprising a metal film to produce the light resonantly reflected from the sensor;
producing a phase shift of at least one wave selected from the group consisting of the reference wave and the measuring wave;
combining the measuring wave and the reference wave to produce a interference pattern; and
recording the interference pattern to determine the spatial phase variation of the light resonantly reflected from the sensor.

34. The method of claim 32 wherein the wave is the reference wave.
